(19) United States
(12) Patent Application Publication
(10) Pub. No.: US 2006/0054501 A1
(43) Pub. Date: Mar. 16, 2006

(54) Methods and apparatus for the measurement of hydrogen sulphide and thiols in fuids

(76) Inventors: Li Jiang, Ridgefield, CT (US); Timothy Gareth John Jones, Cottenham (GB); Nathan Lawrence, Westham (GB); Mary Thompson, Howden (GB); Richard Compton, Oxford (GB)

Correspondence Address: SCHLUMBERGER-DOLL RESEARCH, 36 OLD QUARRY ROAD, RIDGEFIELD, CT, 06877-4108, US

(21) Appl. No.: 10/522,350

(22) PCT Filed: Jul. 10, 2003
(86) PCT No.: PCT/GB03/02991

§ 371 Date: Oct. 20, 2005

(30) Foreign Application Priority Data
Jul. 25, 2002 (GB) 0217249.2

(51) Int. Cl. G01N 33/24

(52) U.S. Cl. 204/409; 436/28

(57) ABSTRACT

An electrochemical sensor for measuring the amount of hydrogen sulphide or thiols in a fluid comprises an electrically conductive porous electrode within which are dispersed a precursor and a reaction solution which together with the hydrogen sulphide or thiols a redox reaction resulting in an electrical current dependent upon the amount of hydrogen sulphide or thiols in the fluid. The reaction solution may be initially provided in the pores of the porous electrode, or derived in use from the fluid itself.

### BACKGROUND TO THE INVENTION

This invention relates to methods and apparatus for measuring the amount of hydrogen sulphide and thiols in fluids, and is more particularly but not exclusively concerned with methods and apparatus for measuring the amount of hydrogen sulphide and thiols in formation fluids from an earth formation surrounding a wellbore.

It is highly desirable to be able to determine at as early a stage as possible the amount of hydrogen sulphide in oil and gas deposits in the earth formations surrounding a wellbore, since the amount of hydrogen sulphide can seriously impact the economic value of the deposits, and affect the composition, and therefore the cost, of the metalwork used in the extraction of the deposits from the formations. Additionally, because hydrogen sulphide is toxic in even relatively low concentrations, the hydrogen sulphide content of the deposits has an important bearing on the health, safety and environmental aspects of their extraction.

Several methods and apparatuses for the measurement of the hydrogen sulphide content of wellbore fluids are described in International Application No. WO 01/63094 (now granted as UK Patent No. 2,395,631). Among these are a method and apparatus based on an electrochemical sensor in which the current created by a redox reaction involving the hydrogen sulphide is measured. More specifically, the sensor comprises a reaction chamber or cell containing a precursor or catalyst (hereinafter referred to simply as a precursor) in an aqueous reaction solution, the walls of the chamber including a gas permeable membrane over which the wellbore fluids flow and through which hydrogen sulphide in the wellbore fluids diffuses into the reaction chamber to initiate the redox reaction.

It is an object of the present invention to provide improved electrochemical sensors of the type in which the current created by a redox reaction involving the hydrogen sulphide is measured.

### SUMMARY OF THE INVENTION

According to a first aspect of the present invention, there is provided an electrochemical sensor for measuring the amount of hydrogen sulphide or thiols in a fluid, the sensor comprising containment means which is adapted to receive the hydrogen sulphide or thiols from the fluid and which contains a precursor and a reaction solution which together with the hydrogen sulphide or thiols create a redox reaction resulting in an electrical current dependent upon the amount of hydrogen sulphide or thiols in said fluid, wherein the containment means comprises an electrically conductive porous member in which said precursor and said reaction solution are dispersed.

It will be appreciated that by replacing the reagent-containing reaction chamber and gas permeable membrane of the aforementioned International Application with a porous member within which the reagents are dispersed, the sensor of the present invention can be miniaturized, which leads to faster response times, lower consumption of reagents and lower unit costs.

In a preferred implementation of the invention, the porous member is moulded from a mixture of the precursor in powder form and a suitable binder also in powder form, and the reaction solution is dispersed in the pores of the porous member. Thus the precursor may be selected from, by way of non-limitative example, N,N′-diphenyl-1,4-phenylenediamine, N,N′dimethylphenyl-1,4-diamine, catechol and dopamine, while the binder may be an epoxy resin, such as a 12% hardener Durcisseur MA2. Additionally, the mixture may further include a powder conductivity agent, such as metal powder or carbon powder. Advantageously, the binder, the precursor and the conductivity agent are mixed in proportions of about 1:1.4:1.6 by weight.

The reaction solution is preferably acidic, and may for example be dilute hydrochloric acid. Also, the reaction solution may include a gelling agent, for example a cross-linked water-soluble polymer such as polyacrylamide and a cross-linking agent such as formaldehyde or N,N′-methylenebisacrylamide, to substantially immobilize the solution in the porous member.

Optionally, the porous member may be adapted to receive the hydrogen sulphide or thiols from the wellbore fluid via a permeable membrane provided on one face thereof.

Conveniently, the porous member serves as a working electrode, and the sensor further comprising a counter electrode and a reference electrode spaced apart but in contact with the porous member, whereby, in use, said current flows between the working and counter electrodes.

The means for measuring said current preferably comprises a working electrode, a counter electrode and a reference electrode, the electrodes being spaced apart but in contact with the porous member, and means for measuring the current flowing between the working and counter electrodes.

From another aspect, the invention also includes a method of measuring the amount of hydrogen sulphide or thiols in formation fluid from an earth formation surrounding a wellbore, the method comprising positioning a wellbore tool equipped with an electrochemical sensor in accordance with the first aspect of the invention in the wellbore adjacent to the formation, exposing the sensor to the formation fluid, and measuring the resulting redox current produced by the sensor.

In yet another aspect, the invention provides an electrochemical sensor for measuring the amount of hydrogen sulphide or thiols in a fluid, the sensor comprising containment means which is adapted to receive the hydrogen sulphide or thiols from the fluid and which contains a precursor which together with the hydrogen sulphide or thiols and a reaction solution create a redox reaction resulting in an electrical current dependent upon the amount of hydrogen sulphide or thiols in said fluid, wherein the containment means comprises an electrically conductive porous member in which said precursor is dispersed.

In a preferred embodiment of this aspect of the invention, the porous member is deposited on a non-conductive base and serves as a working electrode, the reaction solution is derived, in use, from said fluid, and the sensor further comprises a reference electrode and a counter electrode also deposited on said base, whereby, in use, said current flows between the working and counter electrodes. In this embodiment, said counter electrode preferably comprises platinum, said reference electrode preferably comprises silver/silver chloride, and said electrodes are preferably deposited by screen printing. If desired, the electrodes may be covered by a permeable membrane, to protect them from contaminants in the fluid.

The base may be made from a plastics material.

The precursor is advantageously selected from N,N′-diphenyl-1,4-phenylenediamine, N,N′dimethylphenyl-1,4-diamine, catechol and dopamine.

The porous member preferably includes a powder conductivity agent, such as metal powder or carbon powder, to render it conductive.

In a further aspect, the invention concerns the use of an electrochemical sensor as defined above for measuring the amount of hydrogen sulphide or thiols in a fluid, downhole, during logging. The logging is implemented while drilling or is a wireline drilling. The use of the electrochemical sensor may be implemented at the surface or for measuring the amount of hydrogen sulphide or thiols of an underground aquifer.

### BRIEF DESCRIPTION OF THE DRAWINGS

The invention will now be described, by way of example only, with reference to the accompanying drawings, of which:

FIG. 1 is a somewhat schematic representation of a wellbore tool which is positioned in a wellbore and which is equipped with an electrochemical sensor in accordance with the present invention for measuring the amount of hydrogen sulphide or thiols in formation fluid from an earth formation surrounding the wellbore;

FIG. 2 is a schematic representation of the electrochemical sensor of FIG. 1;

FIG. 3 shows some cyclic voltammograms for the sensor of FIG. 1 for various concentrations of hydrogen sulphide;

FIG. 4 shows an alternative embodiment of an electrochemical sensor in accordance with the present invention for measuring the amount of hydrogen sulphide or thiols in a fluid; and

FIG. 5 shows some cyclic voltammograms for the sensor of FIG. 4 for various concentrations of hydrogen sulphide.

### DETAILED DESCRIPTION OF THE INVENTION

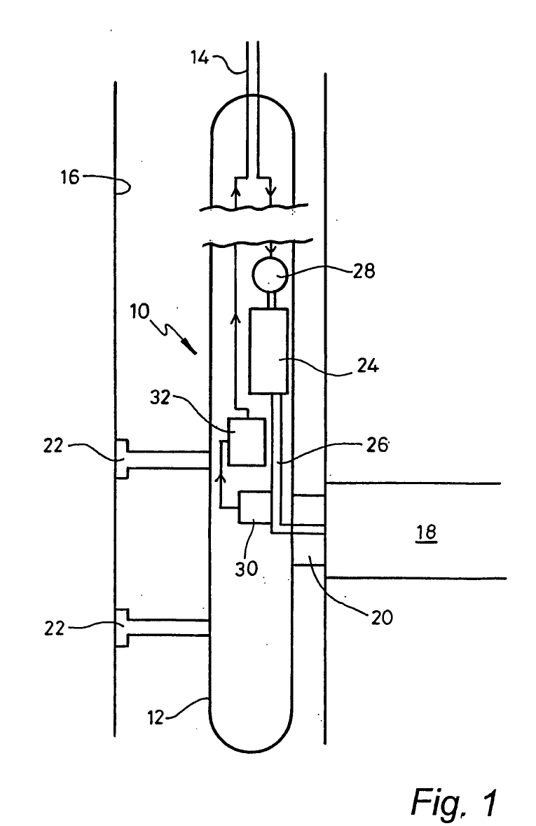

The wellbore tool shown in FIG. 1 is indicated at 10, and is based on Schlumberger's well known modular dynamics tester, as described in Trans. SPWLA 34^{th} Annual Logging Symposium, Calgary, June 1993, Paper ZZ and in U.S. Pat. Nos. 3,780,575, 3,859,851 and 4,994,671. The tool 10 comprises an elongate substantially cylindrical body 12, which is suspended on a wireline 14 in the wellbore, indicated at 16, adjacent an earth formation 18 believed to contain recoverable hydrocarbons, and which is provided with a radially projecting sampling probe 20. The sampling probe 20 is urged into firm contact with the formation 18 by hydraulically operated rams 22 projecting radially from the body 12 on the opposite side from the sampling probe, and is connected internally of the body to a sample chamber 24 by a conduit 26.

In use, and prior to completion of the well constituted by the wellbore 16, a pump 28 within the body 12 of the tool 10 draws a sample of the hydrocarbons into the sample chamber 24 via the conduit 26. The pump is controlled from the surface at the top of the wellbore via the wireline 14 and control circuitry (not shown) within the body 12. It will be appreciated that this control circuitry also controls valves (not shown) for selectively routing the sampled hydrocarbons either to the sample chamber 24 or to a dump outlet (not shown), but these have been omitted for the sake of simplicity.

In accordance with the present invention, the conduit 26 also communicates with an electrochemical sensor 30 also provided within the body 12 of the tool 10, so that the hydrocarbons flow over a face of the sensor on their way through the conduit. As will become apparent, the sensor 30 produces an output current which is dependent on the amount of hydrogen sulphide or thiols in the sampled hydrocarbons. This output current is measured in known manner by a digital current measuring circuit 32 in the body 12 of the tool 10, and the measurement is transmitted to the surface via the wireline 14.

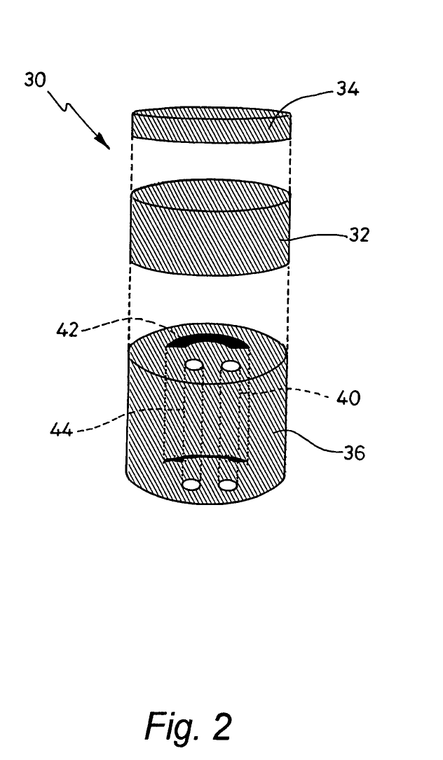

The sensor 30 is shown in an exploded view in FIG. 2, and comprises a generally cylindrical moulded porous block 32 having a circular gas permeable membrane 34 coaxially secured to one end, and a generally cylindrical electrode block 36 coaxially secured to the other end. The porous block 32, the gas permeable membrane 34 and the electrode block 36 are mounted in an open-ended cylindrical housing (not shown), with the membrane 34 facing out of the open end of the housing, and the housing itself is adapted to be secured to an opening or port in the conduit 26 of FIG. 1 so that the sampled hydrocarbons flow over the exposed face of the membrane.

The porous block 32 is moulded from a mixture of a powdered epoxy resin binder in the form of a 12% hardener Durcisseur MA2, a powdered precursor in the form of N,N′-diphenyl-1,4-phenylenediamine and powdered carbon, in the proportions 1.0:1.4:1.6 by weight. Other proportions are possible, such as 1.0:1.0:2.0, depending on the concentration range of the hydrogen sulphide or thiols. For example, experiments have shown that the proportions 1.0:1.4:1.6 provide optimum sensitivity to hydrogen sulphide in concentrations in the range 0.7 ppm to 3.5 ppm.

The electrode block 36 is moulded integrally with the porous block 32 of the same powdered materials, but has three electrodes moulded into it, a working electrode 40, a counter electrode 42 and a reference electrode 44. Dispersed throughout the pores of the blocks 32, 36 is an aqueous reaction solution in the form of 0.1 molar hydrochloric acid, which is preferably immobilized by gelling it with a dilute solution, about 7 g/litre, of a crosslinked water-soluble polymer, such as polyacrylamide of mean (weight averaged) molecular weight 2-5×10⁶ g/mole using formaldehyde or N,N′-methylenebisacrylamide as a crosslinking agent.

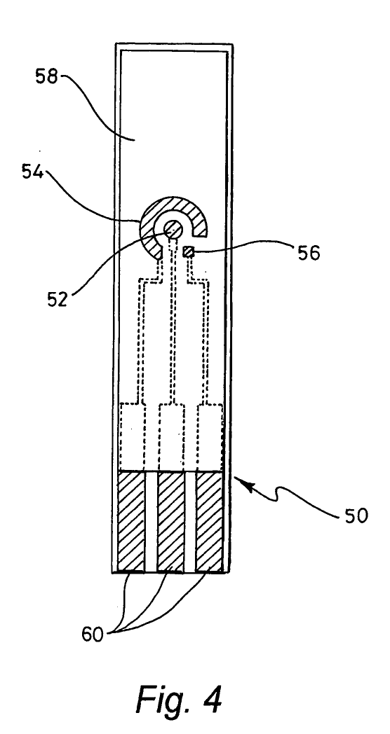

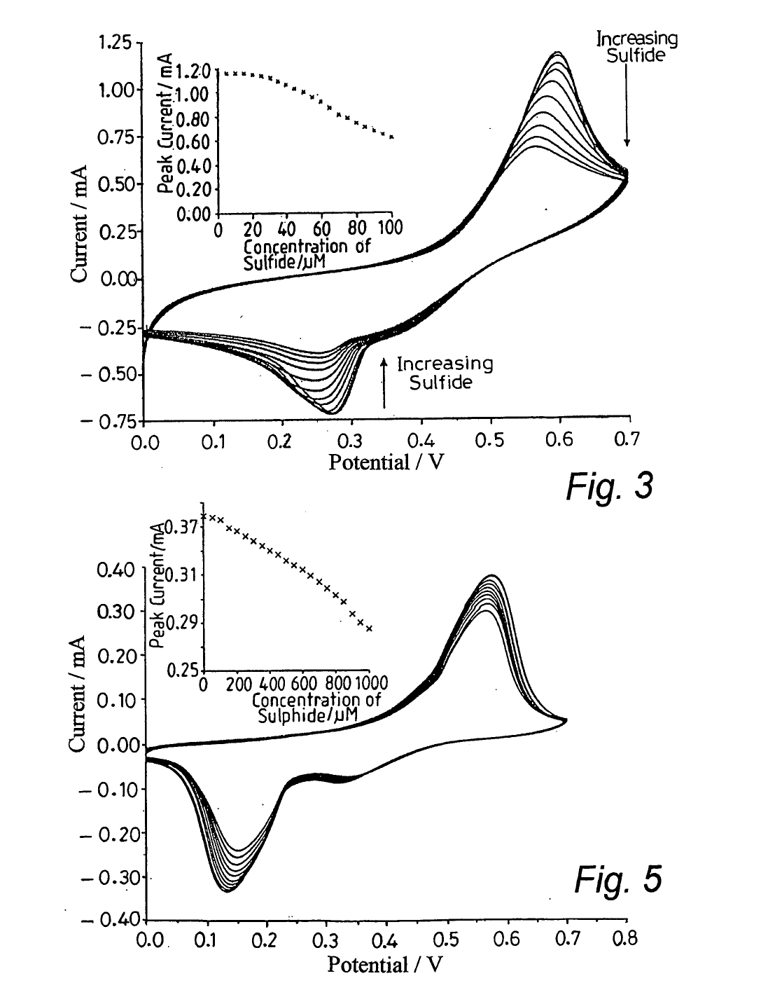

In operation, while the sensor 30 is exposed to the sampled hydrocarbons, suitable electronic measurement equipment is used to apply a cyclically varying potential between the working electrode 40 and the reference electrode 44, and to measure the peak oxidation current flowing between the working electrode 40 and counter electrode 42. Cyclic voltammograms for the sensor 30 is shown in FIG. 3, which includes an inset graph showing the variation of the peak oxidation current with sulphide concentration. It can be seen that for concentrations of sulphide between 20×10⁻⁶ molar (0.7 ppm) and 100×10⁶ molar (3.5 ppm), the oxidation current decreases substantially linearly with increasing sulphide concentration.

Many modifications can be made to the described implementations of the sensor 30.

In particular, powdered precursors other than N,N′-diphenyl-1,4-phenylenediamine can be used, for example N,N′dimethylphenyl-1,4-diamine, catechol or dopamine, the latter two being especially suitable for measuring higher concentrations of hydrogen sulphide. For even higher concentrations of hydrogen sulphide, an aqueous solution of ferrocyanide ions, e.g. potassium ferrocyanide at pH=10, can been used.

In another modification, particularly usable when the aqueous reaction solution is immobilized by gelling it, the gas permeable membrane 34 can simply be omitted, since the gelling, in addition to keeping the reaction solution in the porous block 32, also tends to prevent contaminants from entering the block.

The invention has been described so far in relation to an electrochemical sensor which is particularly suitable for making measurements downhole in a wellbore of the amount of hydrogen sulphide and thiols in formation fluids from an earth formation surrounding the wellbore. However, the invention is not limited to electrochemical sensors for downhole use, and an electrochemical sensor suitable for surface use with any fluid, for example sewage effluent, that may contain hydrogen sulphide or thiols is shown in FIG. 4.

Thus the electrochemical sensor of FIG. 4 is indicated at 50, and comprises three electrodes 52, 54, 56 deposited, eg by screen printing, on a plastic substrate or base 58. The electrode 52 is the working electrode, and comprises a porous dot of carbon powder mixed with N,N′-diphenyl-1,4-phenylenediamine, while the electrode 54 is the counter electrode, and comprises a substantially semicircular strip of platinum substantially concentric with the working electrode. The electrode 56 is the reference electrode, and comprises a small dot of silver/silver chloride spaced from the working electrode 52 on substantially the opposite side thereof from the counter electrode 54. The electrodes may if necessary be covered with a protective membrane which is permeable to hydrogen sulphide, to protect them from contamination by contaminants such as sewage particulates or oily films in the fluid whose hydrogen sulphide content is being measured.

Respective conductive tracks (not shown) on the plastic base 58 connect the electrodes 52, 54, 56 to respective electrical contacts 60 on the edge of the plastic base.

In use, the sensor 50 is exposed to the fluid whose hydrogen sulphide or thiol content is to be measured, and the electrodes 52, 54, 56 effectively become surrounded by or immersed in the fluid: since the fluid is typically electrically conductive, eg by virtue of being aqueous, it acts in a manner analogous to the reaction solution of the sensor 30 of FIG. 2. Accordingly, electronic measurement equipment is connected to the electrodes 52, 54, 56 via the contacts 60, to measure the oxidation current as described in relation to the sensor 30. Cyclic voltammograms for the sensor 50 is shown in FIG. 5, which again includes an inset graph showing the variation of the peak redox current with sulphide concentration. In the case of the sensor 50, it can be seen that for concentrations of sulphide in the range 0 to 1×10⁻³ molar (0 to 34 ppm), the oxidation current decreases substantially linearly with increasing sulphide concentration.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

1. An electrochemical sensor for measuring the amount of hydrogen sulphide or thiols in a fluid, the sensor comprising containment means which is adapted to receive the hydrogen sulphide or thiols from the fluid and which contains a precursor and a reaction solution which together with the hydrogen sulphide or thiols create a redox reaction resulting in an electrical current dependent upon the amount of hydrogen sulphide or thiols in said fluid, wherein the containment means comprises an electrically conductive porous member in which said precursor and said reaction solution are dispersed.

2. An electrochemical sensor as claimed in claim 1, wherein the porous member is moulded from a mixture of the precursor and a suitable binder all in powder form, and the reaction solution is dispersed in the pores of the porous member.

3. An electrochemical sensor as claimed in claim 2, wherein the precursor is selected from N,N′-diphenyl-1,4-phenylenediamine, N,N′dimethylphenyl-1,4-diamine, catechol and dopamine.

4. An electrochemical sensor as claimed in claim 2, wherein the binder is an epoxy resin.

5. An electrochemical sensor as claimed in claim 4, wherein the binder is a 12% hardener Durcisseur MA2.

6. An electrochemical sensor as claimed in claim 2, wherein the mixture further includes a powder conductivity agent.

7. An electrochemical sensor as claimed in claim 6, wherein the powder conductivity agent is selected from metal powder and carbon powder.

8. An electrochemical sensor as claimed in claim 6, wherein the binder, the precursor and the conductivity agent are mixed in proportions of about 1:1.4:1.6 by weight.

9. An electrochemical sensor as claimed in claim 1, wherein the reaction solution includes a gelling agent.

10. An electrochemical sensor as claimed in claim 9, wherein the gelling agent is a cross-linked water-soluble polymer.

11. An electrochemical sensor as claimed in claim 10, wherein the gelling agent comprises polyacrylamide and a cross-linking agent selected from formaldehyde and N,N′-methylenebisacrylamide.

12. An electrochemical sensor as claimed in claim 1, wherein the reaction solution is an acidic solution such as dilute hydrochloric acid.

13. An electrochemical sensor as claimed in claim 1, wherein the porous member is adapted to receive the hydrogen sulphide or thiols from the wellbore fluid via a permeable membrane provided on one face thereof.

14. An electrochemical sensor as claimed in claim 1, wherein the porous member serves as a working electrode, and further comprising a counter electrode and a reference electrode spaced apart but in contact with the porous member, whereby, in use, said current flows between the working and counter electrodes.

15. An electrochemical sensor as claimed in claim 1, further comprising means for measuring said current.

16. A method of measuring the amount of hydrogen sulphide or thiols in formation fluid from an earth formation surrounding a wellbore, the method comprising positioning a downhole tool equipped with an electrochemical sensor in accordance with any preceding claim in the wellbore adjacent to the formation, exposing the sensor to the formation fluid, and measuring the current produced by the sensor.

17. An electrochemical sensor for measuring the amount of hydrogen sulphide or thiols in a fluid, the sensor comprising containment means which is adapted to receive the hydrogen sulphide or thiols from the fluid and which contains a precursor which together with the hydrogen sulphide or thiols and a reaction solution create a redox reaction resulting in an electrical current dependent upon the amount of hydrogen sulphide or thiols in said fluid, wherein the containment means comprises an electrically conductive porous member in which said precursor is dispersed.

18. An electrochemical sensor as claimed in claim 17, wherein the porous member is deposited on a non-conductive base, and wherein the reaction solution is derived, in use, from said fluid.

19. An electrochemical sensor as claimed in claim 17 or claim 18, wherein said porous member serves as a working electrode, and further comprising a reference electrode and a counter electrode also deposited on said base, whereby, in use, said current flows between the working and counter electrodes.

20. An electrochemical sensor as claimed in claim 19, wherein said counter electrode comprises platinum.

21. An electrochemical sensor as claimed in claim 19, wherein said reference electrode comprises silver/silver chloride.

22. An electrochemical sensor as claimed in claim 19, wherein said electrodes are deposited by screen printing.

23. An electrochemical sensor as claimed in claim 19, wherein the electrodes are screened from the fluid by a permeable membrane.

24. An electrochemical sensor as claimed in claim 18, wherein said base is made from a plastics material.

25. An electrochemical sensor as claimed in claim 17, wherein the precursor is selected from N,N′-diphenyl-1,4-phenylenediamine, N,N′dimethylphenyl-1,4-diamine, catechol and dopamine.

26. An electrochemical sensor as claimed in claim 17, wherein the porous member includes a powder conductivity agent to render it conductive.

27. An electrochemical sensor as claimed in claim 26, wherein the powder conductivity agent is selected from metal powder and carbon powder.

28. An electrochemical sensor as claimed in claim 1, wherein the sensor measures the amount of hydrogen sulphide or thiols in a fluid, downhole, during logging.

29. An electrochemical sensor as claimed in claim 28, wherein said logging is performed while drilling.

30. An electrochemical sensor as claimed in claim 28, wherein said logging is wireline logging.

31. An electrochemical sensor as claimed in claim 1, wherein the sensor measures the amount of hydrogen sulphide or thiols, at the surface.

32. An electrochemical sensor as claimed in claim 1 wherein the sensor measures the amount of hydrogen sulphide or thiols of an underground aquifer.
